(19) United States
(12) Patent Application Publication
(10) Pub. No.: US 2006/0276438 A1
(43) Pub. Date: Dec. 7, 2006

(54) Prevention and treatment of influenza with glutamine antagonist agents

(76) Inventors: Natarajan Sethuraman, Hanover, NH (US); Joseph Roberts, Columbia, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 11/240,759

(22) Filed: Sep. 30, 2005

Related U.S. Application Data
(60) Provisional application No. 60/615,662, filed on Oct. 4, 2004.

(51) Int. Cl. A61K 31/663; A61K 31/42; A61K 31/366; A61K 31/198

(52) U.S. Cl. 514/114; 514/345; 514/378; 514/564; 514/453

(57) ABSTRACT

A method of preventing or treating influenza or an influenza-related symptom in a subject by administering to the subject a glutamine antagonist agent is described.

### CROSS REFERENCE TO RELATED PATENT APPLICATION

The present invention is a non-provisional of U.S. Provisional Patent Application Ser. No. 60/615,662, filed Oct. 4, 2004, which is hereby incorporated herein by reference in its entirety.

### BACKGROUND OF THE INVENTION

(1) Field of the Invention

The present invention relates to compounds and methods that are useful for the prevention or treatment of viral infections, and more particularly to compounds and methods that are useful for the prevention or treatment of influenza.

(2) Description of the Related Art

Influenza is a highly contagious, acute respiratory disease that affects all age groups and can occur repeatedly in any particular individual. The etiological agent of the disease is the influenza virus. In the United States alone, influenza is responsible for an average of 114,000 hospitalizations and 20,000 deaths each year. Furthermore, highly unpredictable pathogenic strains of the influenza A virus have emerged causing widespread pandemics, such as the one in 1918 that caused the death of 20-40 million people worldwide. In 1997, an avian influenza A (H5N1) virus that was directly transmitted from chickens to humans killed 30% (6 out of 18) of the infected humans.

Influenza viruses are orthomyxoviruses, which are classified as types A, B, or C by complement-fixing antibodies to the nucleoprotein and matrix proteins. Only types A and B are known to cause classic influenza symptoms in humans. Currently only one serologic type of influenza B virus is recognized. However, influenza A viruses have been categorized into subtypes based on direct antigenic divergence of the two principal surface glycoproteins, hemagglutinin (HA) and neuraminidase (NA).

Two classes of drugs are currently licensed in a large number of countries for the treatment of influenza. The M2 ion channel blockers or amantadines (amantadine and rimantadine) are specific inhibitors of influenza A virus replication, whereas the neuraminidase inhibitors (zanamivir and oseltamivir) are active against influenza A and B viruses. Schmidt, A. C., Drugs, 64(18):2031-46 (2004). However, the development of drug resistant strains often limits the effectiveness of the current antiviral therapies. This results from the pandemic strains of the influenza virus usually possessing antigenically different, novel glycoproteins resulting from antigenic shift, which render the available vaccines ineffective. Generation of resistant infectious influenza viruses by reverse genetics in the laboratory has also raised the possibility of the use of these influenza viruses as potential biological warfare agents.

Given the potential danger of a natural epidemic and pandemic of influenza virus along with its potential use as a biological warfare agent, there is an urgent and immediate need to develop new drugs effective against the influenza viruses and to find methods for their effective use. In particular, it would be useful to provide such drugs and methods that are effective against both A and B strains of influenza. It would also be useful if such drugs had acceptable-to-high safety indexes, and demonstrated low toxicity towards host cells. It would also be useful to provide such drugs and methods that can be administered during all stages of influenza infection, even in elderly and immunosuppressed subjects. Moreover, it would be useful if such drugs were of small molecular weight and could be delivered intranasally.

### SUMMARY OF THE INVENTION

Briefly, therefore the present invention is directed to a novel method of preventing or treating influenza or an influenza-related symptom in a subject, the method comprising administering to the subject a glutamine antagonist agent.

The present invention is also directed to the use of a glutamine antagonist agent for the production of a medicament for the prevention or treatment of influenza or an influenza-related symptom in a subject.

Among the several advantages found to be achieved by the present invention, therefore, may be noted the provision of drugs and methods effective against the influenza viruses, the provision of such drugs and methods that are effective against both A and B strains of influenza, the provision of such drugs having acceptable-to-high safety indexes, and demonstrating low toxicity towards host cells, the provision of such drugs and methods that can be administered during all stages of influenza infection, even in elderly and immunosuppressed subjects, and such drugs of small molecular weight which can be delivered intranasally.

### DETAILED DESCRIPTION OF THE PREFERRED EMBODIMENTS

In accordance with the present invention, it has been discovered that influenza or an influenza -related symptom can be treated in a subject by administering to the subject an influenza effective amount of a glutamine antagonist agent. The glutamine antagonist agent of the present invention can be a glutamine analog that interferes with glutamine metabolism, an agent that inhibits glutamine synthesis, such as an inhibitor or glutamine synthase, a glutamine depleting enzyme, an agent that inhibits glutamine uptake by a cell, or a compound that binds glutamine, thereby reducing its biological availability. In particular, it has been found that glutamine analogs, such as acivicin (L-(alpha S,5S)-alpha-amino-3-chloro-4,5-dihydro-5-isoxazoleacetic acid), and DON (6-diazo-5-oxo-L-norleucine), are particularly effective in the novel method. Without being bound to this or any other theory, it is believed that these compounds cause rapid inhibition of viral cell replication and then viral cell death, resulting in faster patient recovery.

The use of glutamine antagonist agents for the treatment of viral infections such as influenza has been found to be particularly effective, because these compounds—unlike currently available influenza drugs—have been shown to be effective against both A and B strains of influenza. Moreover, these drugs have acceptable-to-high safety indexes, and have demonstrated low toxicity towards host cells in previous evaluation in cancer patients. An advantage of the present method is that lower dosages of the glutamine antagonist agents are required for influenza treatment than for cancer treatment—where unpleasant, and even dose limiting, side effects have been reported for higher dosages of these drugs. See, for example, Earhart et al., Cancer Treatment Reports, 66(5):1215-1217 (1982), Jha et al., Internet Electronic Journal of Molecular Design, 2:539-545 (2003), Earhart et al., Investigational New Drugs, 8:113-119 (1990), and Baruchel et al., Investigational New Drugs, 13:211-216 (1995).

Furthermore, the glutamine antagonist agents can be administered during all stages of influenza infection, even in elderly and immunosuppressed subjects. The active compounds are of small molecular weight and can be delivered intranasally.

In certain embodiments, the present method is useful not only for the treatment of influenza, but also for its prevention. For example, the present glutamine antagonist agents can be administered to subjects who, due to genetic or environmental circumstance, may be at risk of contacting influenza, in order to prevent or reduce the likelihood of influenza infection in the treated subject. Dosage rates and methods of administration of the glutamine antagonist agents for the purpose of prevention are the same as those that are described herein for the treatment of influenza.

The active agent of the present invention is a glutamine antagonist agent. The glutamine antagonist agent is a compound that interferes with the synthesis or use of glutamine in a cell. In preferred embodiments, the glutamine antagonist agent interferes with the synthesis or use of glutamine in a living cell, and preferably in a cell that is part of a living organism—namely, in vivo. When it is said that the glutamine antagonist agent interferes with the synthesis of glutamine, it is meant that the agent acts to reduce the amount or rate of glutamine synthesis to less than the amount or rate that would be experienced in the absence of the glutamine antagonist agent. When it is said that the glutamine antagonist agent interferes with the use of glutamine, it is meant that the agent acts to inhibit or block a metabolic pathway downstream of glutamine, that is, a pathway in which glutamine acts as a precursor of one or more non-glutamine compounds, or that the agent acts to deplete glutamine in a cell or an organism by reacting the glutamine to form a non-glutamine product, or by reversibly or irreversibly binding with glutamine to reduce its availability.

The glutamine antagonist agent of the present invention can be a glutamine analog that interferes with a glutamine metabolic pathway, an agent that inhibits the synthesis of glutamine, a glutamine depleting enzyme, a compound that reacts with glutamine under intracellular conditions to form a non-glutamine product, an agent that inhibits glutamine uptake by cells, or a glutamine binding compound that reduces the biological availability of glutamine. It should be recognized that a compound that is a useful glutamine antagonist agent may have two or more of these characteristic. For example, a compound that is a glutamine analog that interferes with a glutamine metabolic pathway might also act as an agent that inhibits the synthesis of glutamine.

The glutamine antagonist agent can be a glutamine analog that interferes with a glutamine metabolic pathway. Examples of compounds that can act in this manner include acivicin (L-(alpha S,5S)-alpha-amino-3-chloro-4,5-dihydro-5-isoxazoleacetic acid), DON (6-d iazo-5-oxo-L-norleucine), azaserine, azotomycin, chloroketone (L-2-amino-4-oxo-5-chloropentanoic acid), N³-(4-methoxyfumaroyl)-L-2,3-diaminopropanoic acid (FMDP) (inactivates glucosamine-6-phosphate synthase (EC 2.6.1.16), See, Zgòdka et al., Microbiology, 147:1955-1959 (2001)), (3S,4R)-3,4-dimethyl-L-glutamine, (3S,4R)-3,4-dimethyl-L-pyroglutamic acid (See, Acevedo et al., Tetrahedron., 57:6353-6359 (2001)), 1,5-N,N′-disubstituted-2-(substituted benzenesulphonyl) glutamamides (See, Srikanth et al., Bioorganic and Medicinal Chemistry, ______ (2002)), or a mixture of any two or more of these.

The glutamine antagonist agent can be an agent that inhibits the synthesis of glutamine. Examples of compounds having this activity include inhibitors of glutamine synthase (EC 6.3.1.2), such as L-methionine-DL-sulfoximine, and phosphinothricin; inhibitors of glutamate synthase (EC 1.4.1.13); and inhibitors of amidophosphoribosyltransferase (EC 2.4.2.14), and mixtures of any two or more of these.

The glutamine antagonist agent can be a glutamine depleting enzyme. Examples of such enzymes include carbamoyl-phosphate synthase (EC 6.3.5.5), glutamine-pyruvate transaminase (EC 2.6.1.15), glutamine-tRNA ligase (EC 6.1.1.18), glutaminase (EC 3.5.1.2), D-glutaminase (EC 3.5.1.35), glutamine N-acyltransferase (EC2.3.1.68), glutaminase-asparaginase (in particular glutaminase-asparaginase of Pseudomonas 7a and Acinatobacter sp.), and mixtures of any two or more of these.

The glutamine antagonist agent can be a compound that reacts with glutamine under intracellular conditions to form a non-glutamine product. An example of a compound having this property is phenylbutyrate (See Darmaun et al., Phenylbutyrate-induce glutamine depletion in humans: effect on leucine metabolism, pp. E801-E807, in Glutamine Depletion and Protein Catabolism, Am. Physiol. Soc. (1998)). Another example of a glutamine antagonist agent having this characteristic is phenylacetate (See, U.S. Pat. No. 6,362,226).

The glutamine antagonist agent can be an agent that inhibits glutamine uptake by cells. Examples of compounds having this property include alpha-methylaminoisobutyric acid (inhibits GynT plasma membrane glutamine transporter; See, Varoqui et al., J. Biol. Chem., 275(6):4049-4054 (2000), wortmannin, and LY-294002 (inhibits hepatic glutamine transporter; See, Pawlik et al., Am. J. Physiol. Gastrointest. Liver Physiol., 278:G532-G541 (2000)).

The glutamine antagonist agent can be a glutamine binding compound that reduces the biological availability of glutamine.

In the present invention, a composition comprising a glutamine antagonist agent is administered to a subject according to standard routes of drug delivery that are well known to one of ordinary skill in the art.

Each of the glutamine antagonist agents of the present invention can be supplied in the form of a salt, or prodrug, if desirable. Glutamine antagonist agents that are useful in the present invention can be of any purity or grade, but it is preferred that the agent be of a quality suitable for pharmaceutical use. The glutamine antagonist agent can be provided in pure form, or it can be accompanied with impurities or commonly associated compounds that do not affect its physiological activity or safety.

The glutamine antagonist agents can be supplied in the form of a pharmaceutically active salt, a prodrug, an isomer, a tautomer, a racemic mixture, or in any other chemical form or combination that, under physiological conditions, still provides for modulation of a glutamine metabolic pathway, the inhibition of glutamine synthesis, the depletion of glutamine from the body of the subject, inhibition of the uptake of glutamine into cells, or binds to glutamine to decrease its biological availability. The present invention includes all possible diastereomers as well as their racemic and resolved, enantiomerically pure forms.

The compounds useful in the present invention can have no asymmetric carbon atoms, or, alternatively, the useful compounds can have one or more asymmetric carbon atoms. When the useful compounds have one or more asymmetric carbon atoms, they, therefore, include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention. Also included in the methods, combinations and compositions of the present invention are the tautomeric forms of the described compounds.

Also included in the methods and compositions of the present invention are the prodrugs of the described compounds and the pharmaceutically acceptable salts thereof. The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe.

The term “pharmaceutically acceptable” is used adjectivally herein to mean that the modified noun is appropriate for use in a pharmaceutical product.

The compounds of the present invention can also be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the glutamine antagonist agents of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, y-hydroxybutyric, galactaric and galacturonic acids.

Exemplary pharmaceutically acceptable salts include the salts of hydrochloric acid and trifluoroacetic acid. All of the above salts can be prepared by those skilled in the art by conventional means from the corresponding compound of the present invention.

In another embodiment of the present invention, the glutamine antagonist agent can be provided in a “pharmaceutically acceptable carrier” or “pharmaceutically acceptable excipient”, both of which are used interchangeably herein, to form a pharmaceutical composition. Thus, in one embodiment, the present invention encompasses a pharmaceutical composition comprising a glutamine antagonist agent and a pharmaceutically acceptable carrier.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. The pharmaceutically acceptable carrier can also be selected on the basis of the desired route of administration of the compound. For example, in a preferred embodiment the carrier is suitable for oral administration.

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

Other pharmacologically active substances can also be present, including other compounds of the present invention. The pharmaceutical compositions of the invention can be prepared by any of the well-known techniques of pharmacy, such as by admixing the components.

The glutamine antagonist agent can be administered by any conventional means available for use in conjunction with pharmaceuticals, either as an individual therapeutic compound or as part of a combination of therapeutic compounds or as a single pharmaceutical composition or as independent multiple pharmaceutical compositions.

Pharmaceutical compositions according to the present invention include those suitable for oral, inhalation spray, rectal, topical, buccal (e.g., sublingual), or parenteral (e.g., subcutaneous, intramuscular, intravenous, intrathecal, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

The compositions of the present invention can be administered enterally, by inhalation spray, rectally, topically, buccally or parenterally in dosage unit formulations containing conventional nontoxic pharmaceutically acceptable carriers, adjuvants, and vehicles as desired. Parenteral administration includes subcutaneous, intramuscular, intradermal, intramammary, intravenous, and other administrative methods known in the art. Enteral administration includes solution, tablets, sustained release capsules, enteric-coated capsules, and syrups. When administered, the pharmaceutical composition may be at or near body temperature.

In certain embodiments, it is preferred that the glutamine antagonist agent is administered by a route that avoids, minimizes, or reduces a toxic effect of the drug. By way of example, it is known that DON results in gastrointestinal (GI) toxicity at levels that are therapeutically effective if administered orally. Consequently, nasal administration of this drug can reduce the GI toxicity, and is a preferred route.

The compounds of the present invention can be delivered orally either in a solid, in a semi-solid, or in a liquid form. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol an hydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin. Solutions and suspensions may be prepared from sterile powders or granules having one or more pharmaceutically acceptable carriers or diluents, or a binder such as gelatin or hydroxypropylmethyl cellulose, together with one or more of a lubricant, preservative, surface active or dispersing agent.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of a muscarinic receptor antagonist, the dose range may be from about 0.01 mg to about 5,000 mg or any other dose, dependent upon the specific modulator, as is known in the art. When in a liquid or in a semi-solid form, the combinations of the present invention can, for example, be in the form of a liquid, syrup, or contained in a gel capsule (e.g., a gel cap). In one embodiment, when a muscarinic receptor antagonist is used in a combination of the present invention, the muscarinic receptor antagonist can be provided in the form of a liquid, syrup, or contained in a gel capsule. In another embodiment, when a glutamine antagonist agent is used in a combination of the present invention, the glutamine antagonist agent can be provided in the form of a liquid, syrup, or contained in a gel capsule.

Oral delivery of the glutamine antagonist agents of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods and compositions of the present invention, the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing the glutamine antagonist agent may be formulated with sweetening agents, for example glycerol, sorbitol, or sucrose. Such formulations may also contain a demulcent, a preservative, and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

The subject method of prescribing a glutamine antagonist agent and compositions comprising the same can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents, which have been mentioned above or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Administration of the glutamine antagonist agent can also be by inhalation, in the form of aerosols or solutions for nebulizers. Therefore, in one embodiment, the glutamine antagonist agent is administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Aerosols of liquid particles comprising the active materials may be produced by any suitable means, such as inhalatory delivery systems. Nebulizers are commercially available devices, which transform solutions, or suspensions of the active ingredient into a therapeutic aerosol mist by means of acceleration of compressed gas, typically either air or oxygen, through a narrow venturi orifice or by means of ultrasonic agitation. Suitable formulations for use in nebulizers consist of the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the active materials may likewise be produced with any solid particulate medicament aerosol generator. Aerosol generators for administering solid particulate medicaments to a subject produce particles, which are respirable, as explained above, and generate a volume of aerosol containing a predetermined metered dose of a medicament at a rate suitable for human administration.

One type of solid particulate aerosol generator is an insufflator. Suitable formulations for administration by insufflation include finely comminuted powders, which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by means of air drawn through the device upon inhalation or by means of a manually operated pump. The powder employed in the insufflator either consists solely of the active ingredient or of a powder blend comprising the active materials, a suitable powder diluent, such as lactose, and an optional surfactant.

A second type of aerosol generator is a metered dose inhaler. Metered dose inhalers are pressurized aerosol dispensers, typically containing a suspension or solution formulation of the glutamine antagonist agent in a liquefied propellant. During use, the metered dose inhaler discharges the formulation through a valve, adapted to deliver a metered volume, to produce a fine particle spray containing the active materials. Any propellant may be used for aerosol delivery, including both chlorofluorocarbon-containing propellants and non-chlorofluorocarbon-containing propellants.

A third type of aerosol generator is a electrohydrodynamic (EHD) aerosol generating device, which has the advantage of being adjustable to create substantially monomodal aerosols having particles more uniform in size than aerosols generated by other devices or methods. Typical EHD devices include a spray nozzle in fluid communication with a source of liquid to be aerosolized, at least one discharge electrode, a first voltage source for maintaining the spray nozzle at a negative (or positive) potential relative to the potential of the discharge electrode, and a second voltage source for maintaining the discharge electrode at a positive (or negative) potential relative to the potential of the spray nozzle. Most EHD devices create aerosols by causing a liquid to form droplets that enter a region of high electric field strength. The electric field then imparts a net electric charge to these droplets, and this net electric charge tends to remain on the surface of the droplet. The repelling force of the charge on the surface of the droplet balances against the surface tension of the liquid in the droplet, thereby causing the droplet to form a cone-like structure known as a Taylor Cone. In the tip of this cone-like structure, the electric force exerted on the surface of the droplet overcomes the surface tension of the liquid, thereby generating a stream of liquid that disperses into a many smaller droplets of roughly the same size. These smaller droplets form a mist, which constitutes the aerosol cloud that the user ultimately inhales.

Administration of the compositions of the present invention can also be rectally. Pharmaceutical compositions suitable for rectal administration are preferably presented as unit-dose suppositories. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Pharmaceutical compositions suitable for topical application to the skin preferably take the form of an ointments, creams, lotions, pastes, gels, sprays, powders, jellies, collyriums, solutions or suspensions, aerosols, or oils. Carriers, which can be used, include petroleum jelly (e.g., Vaseline®), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6):318 (1986).

The compositions of the present invention can optionally be supplemented with additional agents such as, for example, viscosity enhancers, preservatives, surfactants and penetration enhancers.

Viscosity is an important attribute of many medications. Drops that have a high viscosity tend to stay in the body for longer periods and thus, increase absorption of the active compounds by the target tissues or increase the retention time. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives are optionally employed to prevent microbial contamination during use. Suitable preservatives include polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a glutamine antagonist agent topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

Topical delivery systems are also encompassed by the present invention and include ointments, powders, sprays, creams, jellies, collyriums, solutions or suspensions.

Powders have the advantage of sticking to moist surfaces, and consequently, can remain on the skin for long periods. Therefore, powders are especially attractive for certain purulent respiratory disorders.

Pharmaceutically acceptable excipients and carriers encompass all the foregoing and the like. The above considerations concerning effective formulations and administration procedures are well known in the art and are described in standard textbooks. See e.g., Gennaro, A. R., Remington: The Science and Practice of Pharmacy, 20th Edition, (Lippincoft, Williams and Wilkins), (2000); Hoover, John E., Remington's Pharmaceutical Sciences, Mack Publishing Co., Easton Pa., (1975); Liberman, et al., Eds., Pharmaceutical Dosage Forms, Marcel Decker, New York, N.Y., (1980); and Kibbe, et al., Eds., Handbook of Pharmaceutical Excipients (3rd Ed.), American Pharmaceutical Association, Washington, (1999).

Preferably, the present methods and compositions comprise a therapy, which can be used for treating influenza. In particular, the methods and compositions are useful for treating influenza type A, or type B, and are even more useful for treating either type of influenza, in a subject that is in need of the prevention or treatment of this infection, or a symptom thereof.

For purposes of the present invention, it is preferred that the amount of a glutamine antagonist agent that is administered to a subject comprises an effective amount of the treatment agent. Further preferred is that the amount of the glutamine antagonist agent that is administered comprises a therapeutically effective amount.

As used herein, an “effective amount” means the dose or amount to be administered to a subject and the frequency of administration to the subject, which is readily determined by one having ordinary skill in the art, by the use of known techniques and by observing results obtained under analogous circumstances.

In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

As used herein, the terms “therapeutically effective” are intended to qualify the amount of an agent for use in therapy that will achieve the goal of preventing or improving the severity of the disorder being treated, while avoiding adverse side effects typically associated with alternative therapies. Thus, an influenza effective amount is an amount of a glutamine antagonist agent that prevents or improves the severity of influenza, or an influenza symptom, while avoiding, at least to some degree, an adverse side effect typically associated with alternative therapies. Influenza, or a related symptom is considered ameliorated or improved if any benefit is achieved, no matter how slight.

It will be appreciated that the amount of the glutamine antagonist agent required for use in the treatment of influenza will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages.

The appropriate dosage for an influenza effective amount of a glutamine antagonist agent will depend upon the type and activity of the agent. In general, an influenza effect amount is from about 1% to about 100% of the maximum rate limiting dose, and an amount of from about 5% to about 50% of the rate limiting dose is more preferred.

By way of example, if the glutamine antagonist agent is acivicin, an influenza effective amount is from about 0.001 mg per kg of body weight of the subject per day (mg/kg.day) up to about 10 mg/kg.day. A dosage of from about 0.01 mg/kg.day to about 2 mg/kg.day is preferred, a dosage of from about 0.1 mg/kg.day to about 1 mg/kg.day is more preferred, and a dosage of from about 0.1 mg/kg.day to about 0.5 mg/kg.day is yet more preferred. If the glutamine antagonist agent is DON, an influenza effective amount is from about 0.01 mg per kg of body weight of the subject twice weekly up to about 50 mg/kg.twice weekly. A dosage of from about 0.5 mg/kg to about 10 mg/kg twice weekly is preferred, a dosage of from about 1 mg/kg to about 5 mg/kg twice weekly is more preferred, and a dosage of from about 1 mg/kg to about 2 mg/kg twice weekly is yet more preferred. Alternatively, DON can be administered daily in the dosages described above, in multiple doses per day, every other day, every third day, once a week, or the like. In one embodiment, DON is administered in a regimen of administration every day for a certain number of consecutive days followed by no administration for a certain period. An example of such a regimen is administration of DON for five consecutive every four weeks.

The dosage may be administered in single or multiple doses. It is believed that dosages of glutamine antagonist agents at these levels is below the rate that such compounds are normally used in cancer therapy, for example, and therefore, that the dosages that are effective for the treatment of viral infections result in fewer and less severe side effects than such drugs have been found to cause in cancer treatment studies.

As used herein, the term “subject” for purposes of treatment includes any subject, and preferably is a subject who is in need of the treatment of influenza, or who needs treatment of an influenza -related complication. The subject is typically an animal, and yet more typically is a mammal. “Mammal”, as that term is used herein, refers to any animal classified as a mammal, including humans, domestic and farm animals, zoo, sports, or pet animals, such as dogs, horses, cats, cattle, etc. Preferably, the mammal is a human. For purposes of the present invention, an adult human weighs approximately seventy kilograms and has a body surface area of approximately 1.6 m². In certain embodiments, the subject is a non-human animal.

As used herein, the terms “subject is one that is in need of treatment of influenza or an influenza-related complication” refer to any subject who is suffering from influenza or an influenza-related complication described herein. The terms “subject is one that is in need of treatment of influenza or an influenza-related complication” also refer to any subject that requires a lower dose of conventional influenza treatment agents. In addition, the terms “subject is one that is in need of treatment of influenza or an influenza-related complication” means any subject who requires a reduction in the side effects of a conventional influenza treatment agent.

A therapy comprising a glutamine antagonist agent encompasses the treatment of influenza A, B, or C, parainfluenza viruses, and any other influenza-like virus. In particular, the present treatment encompasses the treatment of influenza A or B.

As used herein, the terms “influenza-related symptom” refer to physiological symptoms that are related to an underlying influenza viral infection. Viral infection-related symptoms for influenza, for example, include without limitation, chills, fever, prostration, generalized aches and pains, headache, photophobia, retrobulbur aching, scratchy sore throat, substernal burning, nonproductive cough, and coryza.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### EXAMPLE 1

This example illustrates the antiviral activity of 6-diazo-5-oxo-L-norleucine (DON) against influenza viruses of type A and B.

An antiviral test assay for the determination of the efficacy of DON was carried out according to the methods described in Furata et al., Antimicrobial Agents and Chemotherapy, 46(4):977-981 (2002). The vehicle used was Eagle's modification of minimum essential medium (EMEM). Two tests were carried out with different strains of influenza type A viruses and one test, followed by a confirmatory test, was carried out with an influenza type B virus strain. The cell type in all tests was Madin-Darby canine kidney cells (MDCK), and in all tests drug units and control units are expressed as micrograms/milliliter (μg/ml). The positive control drug in each test was ribavarin. In each test, the concentration of the drug required to exhibit 50% antiviral activity (EC₅₀) was reported, as was the concentration of the drug that showed toxicity for 50% of the MDCK host cells (IC₅₀). The Safety Index (SI) of the drug could then be calculated according to the formula: SI═IC₅₀/EC₅₀. In the confirmatory test, the concentration of the drug required to exhibit 50% antiviral activity (EC₅₀) was reported as well as the concentration of the drug required to exhibit 90% of the antiviral activity (EC₉₀). Also reported was the concentration of the drug that showed toxicity for 50% of the MDCK host cells (IC₅₀). The Safety Index (SI) of the drug could then be calculated according to the formula: SI═IC₅₀/ EC₅₀, or SI═IC₅₀/ EC₉₀. All drug tests were carried out with stationary cells. The results were as follows:

TABLE 1
Efficacy of DON against type A and type B influenza virus.

| | EC₅₀ | EC₉₀ | IC₅₀ | |
|---|---|---|---|---|
| Type of Virus | (μg/ml)^{c} | (μg/ml) | (μg/ml) | SI |
| Influenza A | 0.32 | | >100 | 312 |
| (H1N1; New | 0.21 | | | 476 |
| Caledonia/20/99) | | | | |
| Positive control | 5.5 | | 100 | >18 |
| drug | 5.5 | | | >18 |
| Influenza A | 2.3 | | ≧100 | 43 |
| (H3N2; | 1.8 | | | 56 |
| Panama/2007/99) | | | | |
| Positive control | 3.2 | | >100 | >31 |
| drug | 4.3 | | | >23 |
| Influenza B | 0.032 | | ≧100 | 3,125 |
| (Hong | 0.14 | | | 714 |
| Kong/330/02) | | | | |
| Positive control | 1.8 | | >100 | >56 |
| drug | 2 | | | >50 |
| Influenza B | 0.18 | | >100 | >555 |
| (Hong | | | | |
| Kong/330/02)^{a} | | | | |
| Positive control | 1.8 | | >100 | >56 |
| drug | | | | |
| Influenza B | | 2.5 | >100 | 40^{b} |
| (Hong | | | | |
| Kong/330/02)^{a} | | | | |
| Positive control | | 3.5 | >100 | >29^{b} |
| drug | | | | |

Notes:
^{a}Confirmatory test done under same conditions as initial test with Influenza B strain, but at a later date.
^{b}SI is calculated as IC₅₀/EC₅₀, except where noted as (b), where SI = IC₅₀/EC₉₀.
^{c}In the EC₅₀ test results, the top number is based on CPE inhibition (visual) and the lower number is based on Neutral Red assay. In the confirmatory test with influenza B, activity was measured by the visual method.

The tests showed that DON was active to highly active against all three strains of influenza. In the neutral red assay the toxicity of DON was 25-50% at concentrations of 1-100 μg/ml, indicating some cell inhibitory effects at low concentrations. Tests with VEEV and YFV cells indicated that toxicity by neutral red assay in Vero cells was much greater than in MDCK cells, indicating that the selectivity of the compound may be low.

### EXAMPLE 2

This example illustrates the antiviral activity of acivicin ((alpha-S,5S)-alpha-amino-3-chloro-2-isoxazoline-5-acetic acid) against influenza viruses of type A and B.

An antiviral test assay was carried out according to the methods described in Example 1, except that no confirmatory test was run for influenza B. The results were as follows:

TABLE 2
Efficacy of acivicin against type A and type B influenza virus.

| | EC₅₀ | EC₉₀ | IC₅₀ | |
|---|---|---|---|---|
| Type of Virus | (μg/ml)^{a} | (μg/ml) | (μg/ml) | SI |
| Influenza A | 2.5 | | >100 | >4 |
| (H1N1; New | 1.3 | | | >7.7 |
| Caledonia/20/99) | | | | |
| Positive control | 5.5 | | 100 | >18 |
| drug | 5.5 | | | >18 |
| Influenza A | 50 | | ≧100 | >2 |
| (H3N2; | 55 | | | 1.8 |
| Panama/2007/99) | | | | |
| Positive control | 7 | | >100 | >14 |
| drug | 5.5 | | | >18 |
| Influenza B | 0.32 | | ≧100 | >312 |
| (Hong | 0.32 | | | >312 |
| Kong/330/02) | | | | |
| Positive control | 0.55 | | >100 | >182 |
| drug | 1.2 | | | >83 |

Notes:
^{a}In the EC₅₀ test results, the first number reported is based on CPE inhibition (visual) and the second number is based on Neutral Red. In the confirmatory test with influenza B, activity was measured by the visual method.

The tests showed that acivicin was weakly active to active against influenza virus A (H1N1), not active against influenza virus A (H3N2), and markedly active against influenza virus B.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions by those of ordinary skill in the art without departing from the scope of the invention, it is intended that all matter contained in the above description shall be interpreted as illustrative and not in a limiting sense. In addition it should be understood that aspects of the various embodiments may be interchanged both in whole or in part.

What is claimed is:

1. A method of preventing or treating influenza or an influenza-related symptom in a subject, the method comprising administering to the subject a glutamine antagonist agent.

2. The method according to claim 1, wherein the glutamine antagonist agent comprises:
a glutamine analog that interferes with a glutamine metabolic pathway;
an agent that inhibits the synthesis of glutamine;
a glutamine depleting enzyme;
a compound that reacts with glutamine under intracellular conditions to form a non-glutamine product;
an agent that inhibits glutamine uptake by cells; or a glutamine binding compound that reduces the biological availability of glutamine.

3. The method according to claim 1, wherein the glutamine antagonist agent comprises a glutamine analog that interferes with a glutamine metabolic pathway.

4. The method according to claim 3, wherein the glutamine antagonist agent comprises acivicin (L-(alpha S,5S)-alpha-amino-3-chloro-4,5-dihydro-5-isoxazoleacetic acid), DON (6-diazo-5-oxo-L-norleucine), azaserine, azotomycin, chloroketone (L-2-amino-4-oxo-5-chloropentanoic acid), N³-(4-methoxyfumaroyl)-L-2,3-diaminopropanoic acid (FMDP), (3S ,4R)-3,4-d imethyl-L-glutamine, (3S,4R)-3,4-d imethyl-L-pyrog lutamic acid, 1,5-N, N ′-d isubstituted-2-(substituted benzenesulphonyl) glutamamides, or a mixture of any two or more of these.

5. The method according to claim 1, wherein the glutamine antagonist agent comprises an agent that inhibits the synthesis of glutamine.

6. The method according to claim 5, wherein the glutamine antagonist agent comprises inhibitors of glutamine synthase (EC 6.3.1.2), L-methionine-DL-sulfoximine, phosphinothricin, inhibitors of glutamate synthase (EC 1.4.1.13); inhibitors of amidophosphoribosyltransferase (EC 2.4.2.14), or mixtures of any two or more of these.

7. The method according to claim 1, wherein the glutamine antagonist agent comprises a glutamine depleting enzyme.

8. The method according to claim 7, wherein the glutamine antagonist agent comprises carbamoyl-phosphate synthase (EC 6.3.5.5), glutamine-pyruvate transaminase (EC 2.6.1.15), glutamine-tRNA ligase (EC 6.1.1.18), glutaminase (EC 3.5.1.2), D-glutaminase (EC 3.5.1.35), glutamine N-acyltransferase (EC2.3.1.68), glutaminase-asparaginase, glutaminase-asparaginase of Pseudomonas 7a, glutaminase-asparatinase of Acinatobacter sp, or mixtures of any two or more of these.

9. The method according to claim 1, wherein the glutamine antagonist agent comprises a compound that reacts with glutamine under intracellular conditions to form a non-glutamine product.

10. The method according to claim 10, wherein the glutamine antagonist agent comprises phenylbutyrate or phenylacetate.

11. The method according to claim 1, wherein the glutamine antagonist agent comprises an agent that inhibits glutamine uptake by cells.

12. The method according to claim 1 1, wherein the glutamine antagonist agent comprise alpha-methylaminoisobutyric acid, wortmannin, LY-294002, or mixtures of any two or more of these.

13. The method according to claim 1, wherein the glutamine antagonist agent can be a glutamine binding compound that reduces the biological availability of glutamine.

14. The method according to claim 1, wherein the amount of the glutamine antagonist agent that is administered to the subject is an influenza effective amount.

15. The method according to claim 1, wherein the glutamine antagonist agent is acivicin and is administered to the subject in an amount about 0.001 mg per kg of body weight of the subject per day (mg/kg.day) up to about 10 mg/kg.day.

16. The method according to claim 1, wherein the glutamine antagonist agent is acivicin and is administered to the subject in an amount from about 0.01 mg/kg.day to about 2 mg/kg.day.

17. The method according to claim 1, wherein the glutamine antagonist agent is acivicin and is administered to the subject in an amount from about 0.1 mg/kg.day to about 1 mg/kg.day.

18. The method according to claim 1, wherein the glutamine antagonist agent is acivicin and is administered to the subject in an amount from about 0.1 mg/kg.day to about 0.5 mg/kg.day.

19. The method according to claim 1, wherein the glutamine antagonist agent is DON and is administered to the subject in an amount from about 0.003 mg/kg.day to about 15 mg/kg.day.

20. The method according to claim 1, wherein the glutamine antagonist agent is DON and is administered to the subject in an amount from about 0.5 mg/kg to about 10 mg/kg twice weekly.

21. The method according to claim 1, wherein the glutamine antagonist agent is DON and is administered to the subject in an amount from about 1 mg/kg to about 5 mg/kg twice weekly.

22. The method according to claim 1, wherein the glutamine antagonist agent is DON and is administered to the subject in an amount from about 1 mg/kg to about 2 mg/kg twice weekly.

23. The method according to claim 1, wherein the glutamine antagonist agent is administered by a route that avoids, minimizes, or reduces a toxic effect of the drug.

24. The method according to claim 1, wherein the glutamine antagonist agent is administered intranasally.

25. The use of a glutamine antagonist agent for the production of a medicament for the prevention or treatment of influenza or an influenza-related symptom in a subject.
